(19) United States
(12) Patent Application Publication
(10) Pub. No.: US 2003/0109797 A1
(43) Pub. Date: Jun. 12, 2003

(54) Chromatic display-type biofeedback system and method using brain waves

(76) Inventors: Ki Hong Kim, Taejon (KR); Hyun Bin Kim, Taejon (KR); Ki Ho Kim, Taejon (JP); Jin Sung Choi, Taejon (KR); Hyun Myung, Taejon (KR); Hyuk Jeong, Taejon (KR); Hong Kee Kim, Taejon (KR); Sang Won Ghyme, Jeju (KR); Yong Wan Kim, Taejon (KR); Ki Suk Lee, Seoul (KR)

Correspondence Address: JACOBSON, PRICE, HOLMAN & STERN, PROFESSIONAL LIMITED LIABILITY COMPANY, 400 Seventh Street, N.W., Washington, DC, 20004, US

(21) Appl. No.: 10/067,322

(22) Filed: Feb. 7, 2002

(30) Foreign Application Priority Data
Dec. 11, 2001 (KR) 2001-78057

(51) Int. Cl. A61B 5/04

(52) U.S. Cl. 600/545

(57) ABSTRACT

A chromatic display-type biofeedback system and method using brain waves are disclosed. After the brain waves generated from the subject, which are indicative of anxious or relaxed psychological state, are recoded by himself or herself, the subject transforms each psychological state into a chromatic bar represented by a desired color. And then, when the subject measures his or her brain waves, a specific color is displayed on a monitor, so that the subject may verify the present psychological state and repeatedly remind him or her of a color designated as a relaxed state to induce the reaching of the relaxed state. At that time, after a psychological state of the subject is verified by modeling the brain waves using a Hidden Markov Model (HMM) which is used in a statistical signal process, the results is displayed as a chromatic bar designated by the subject on a monitor, so that conversion of the relevant psychological state can be rapidly and easily achieved.

### BACKGROUND OF THE INVENTION

1. Field of the Invention

The present invention relates to a chromatic display-type biofeedback system and method using brain waves, and more particularly, to a chromatic display-type biofeedback system and method using brain waves, the method comprising the steps of recoding the brain waves generated from a user or subject having various psychological states, transforming each of the brain waves into a specific color which is desired by the subject and storing the colors, acquiring and perceiving the brain waves through a headband-type interface for collecting brain waves in order to verify the psychological state of the subject, and displaying the results as an arbitrary color bar through a display appliance so that the subject identifies his psychological state.

2. Background of the Related Art

Generally, a biofeedback is to set a subject's mind in rest by detecting his brain waves by use of an electroencephalograph. There are proposed several instruments for executing the biofeedback by detecting a physiological reaction, without using expensive precision medical equipments detecting the brain waves directly. Such an instrument detects a galvanic skin response (GSR), a skin temperature, a pulse rate or the like, which are used in a common medical equipment, in order to execute the biofeedback.

The brain waves measured from a skull of a subject have a wavelength containing potential difference of several tens micro-volts and frequencies of below 30 Hertz, and is biological signals reflecting an aware state of humans. The brain waves are sorted into α-wave, β-wave, θ-wave, δ-wave and the like, depending upon the frequency.

The β-wave contains frequencies between 8 to 13 Hertz, and is in connection with a relaxed creative state. The θ-wave contains frequencies between 4 to 8 Hertz, and is often found in teenagers having learning disability. The δ-wave contains frequencies between 0.5 to 4 Hertz, and is typically generated during a deep-deep sleep state. In particular, since the α-wave band brain wave is known to stabilize the psychological state and encourage the attention, studies for increasing an appearance ratio of the α-wave have been progressed.

Since the biofeedback of the brain waves generated as described above informs the subject of the present psychological state, the subject can learn the method of adjusting the brain waves to a target level by himself or herself. In other words, according to the method, the feedback is executed by viewing a screen displaying the state of his or her brain waves or by hearing a sound informing of the reaching of the target level. A biofeedback therapist carefully observes the state and brain waves of the subject to properly adjust the feedback provided to the subject.

The system of encouraging the attention of the subject using the brain waves has been developed. The α-wave, which is easily detected from the brain waves of an attended person, is applied to the subject, to cause the attention to be improved. There is a drawback that the complicated brain waves generated from the attended person are merely explained by the α-wave. In addition, there is no mentioned on other psychological state except the attention.

### SUMMARY OF THE INVENTION

Accordingly, the present invention is directed to a chromatic display-type biofeedback system and method using brain waves that substantially obviates one or more problems due to limitations and disadvantages of the related art.

An object of the present invention is to provide a chromatic display-type biofeedback system and method using brain waves, in which in case of trying to relax a psychological state of a subject, the subject views a color indicative of the present psychological state of the subject by verifying the brain waves directly, so that the subject can attempt to convert it to a specific color indicative of a relaxed state.

To achieve the object and other advantages, the present invention comprises the steps of: recording brain waves generated from the subject by himself or herself, which are indicative of anxious or relaxed psychological state; transforming each psychological state into a chromatic bar represented by a desired color; measuring his or her brain waves; displaying a specific color on a monitor to verify the present psychological state; and repeatedly reminding him or her of a color designated as a relaxed state to induce the reaching of the relaxed state. At that time, after a psychological state of the subject is verified by modeling the brain waves using a Hidden Markov Model (HMM) which is used in a statistical signal process, the results is displayed as a chromatic bar designated by the subject on a monitor, so that conversion of the relevant psychological state can be rapidly and easily achieved.

According to one aspect of the present invention, there is provided a chromatic display-type biofeedback method using brain waves, the method comprising the steps of: measuring and storing the brain waves of a subject to verify a psychological state of the subject; forming a chromatic bar by designating each psychological state of a brain waves pattern related with a specific psychological state of the subject as a desired color; establishing a reference model according to a plurality of psychological states of the subject; dividing a brain waves data measured at a specific psychological state of the subject at a frame of a certain dimension, and extracting a feature information from each frame; comparing the extracted feature information with the preset feature information of the brain waves related with a reference model according to the psychological states; and selecting one model indicative of the psychological state of the subject for the feature information similar to each other, and displaying a previously stored chromatic bar information corresponding to the mode.

The reference model establishing step comprises the steps of: measuring the brain waves to verify the psychological state of the subject; dividing the measured brain waves data at a unit of a frame of a desired dimension, and extracting a parameter value for a feature information from each frame; and sorting each parameter having an analogous value among the parameters extracted from each frame to construct the brain wave pattern according to the psychological state, and establishing a reference model according to each brain waves pattern. The parameter is an autoregressive parameter.

The reference model establishing step comprises the steps of: sorting each parameter having an analogous value among the parameters extracted from each frame as a single group to form a code book; and establishing a Hidden Markov Model by use of each code series obtained by applying the code book to a training data. The code book is established by a vector quantization method using a K-means algorism.

According to another aspect of the present invention, there is provided a chromatic display-type biofeedback system using brain waves, the system comprising: a brain waves measuring unit for measuring the brain waves to verify a psychological state of a subject; a first storing unit for storing a chromatic bar formed by designating each psychological state of a brain waves pattern related with a specific psychological state of the subject as a desired color; a second storing unit for storing reference models related with a plurality of psychological states of the subject; and a feedback signal generating unit for a) dividing a brain waves data measured at a specific psychological state of the subject at a frame of a certain dimension, extracting a feature information from each frame, and comparing the extracted feature information with the storing reference models related with a plurality of psychological states stored in the second storing unit, and b) selecting one model indicative of the psychological state of the subject for the feature information similar to each other, and displaying a previously stored chromatic bar information corresponding to the mode.

The feedback signal generating unit includes: a parameter extracting section for dividing the measured brain waves data at a unit of a frame of a desired dimension, and extracting a parameter value for a feature information from each frame; and a model establishing section for sorting each parameter having an analogous value among the parameters extracted from each frame as a single group through a vector quantization method using a K-means algorism to form a code book, and establishing a Hidden Markov Model by use of each code series obtained by applying the code book to a training data.

According to still another aspect of the present invention, there is provided a storage medium for executing a chromatic display-type biofeedback method using brain waves, the storage medium capable of being read by a digital processor, and storing a program of commands executed by the digital processor, the program being implemented by types, with the program comprising the steps of: measuring and storing the brain waves of a subject to verify a psychological state of the subject; forming a chromatic bar by designating each psychological state of a brain waves pattern related with a specific psychological state of the subject as a desired color; establishing a reference model according to a plurality of psychological states of the subject; dividing a brain waves data measured at a specific psychological state of the subject at a frame of a certain dimension, and extracting a feature information from each frame; comparing the extracted feature information with the preset feature information of the brain waves related with a reference model according to the psychological states; and selecting one model indicative of the psychological state of the subject for the feature information similar to each other, and displaying a previously stored chromatic bar information corresponding to the mode.

It is to be understood that both the foregoing general description and the following detailed description of the present invention are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this application, illustrate embodiment(s) of the invention and together with the description serve to explain the principle of the invention. In the drawings:

FIG. 1 is a schematic view illustrating a chromatic display-type biofeedback system using brain waves according to one preferred embodiment of the present invention;

FIG. 2 is a block diagram illustrating the construction of the brain waves measuring unit of FIG. 1;

FIG. 3 is a block diagram illustrating the construction of the biofeedback unit embedded in the computer system of FIG. 1; and

FIG. 4 is a flowchart of a chromatic display-type biofeedback method using the brain waves according to the present invention.

### DETAILED DESCRIPTION OF THE PREFERRED EMBODIMENTS

The chromatic display-type biofeedback system and method using brain waves according to one preferred embodiment of the present invention will now be explained with reference to the accompanying drawings.

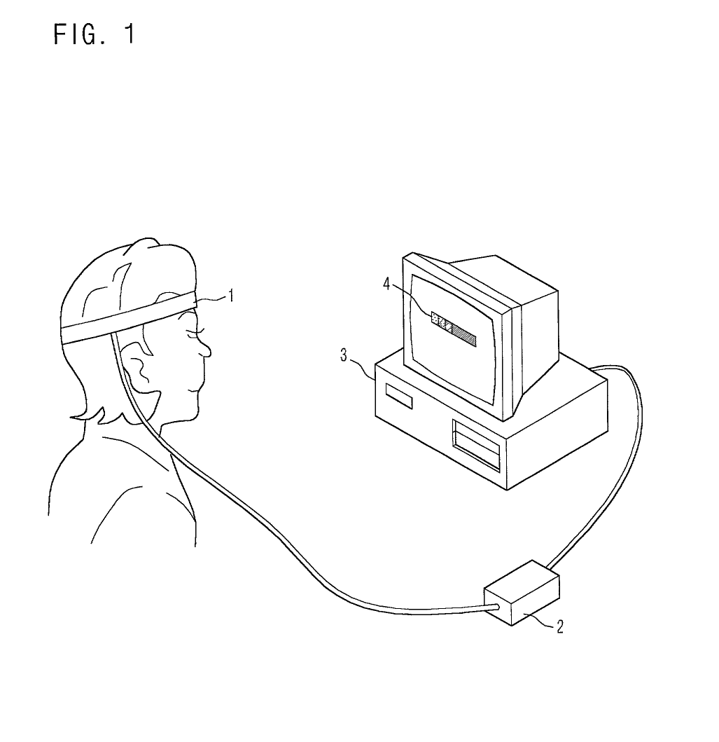

FIG. 1 is a schematic view illustrating a chromatic display-type biofeedback system using brain waves according to one preferred embodiment of the present invention.

The system of the present invention comprises a unit 1 for measuring brain waves of a user or subject, an interfacing unit 2 for amplifying the brain waves measured by the unit 1, i.e., a voltage of several tens micro-volts to a voltage of a few volts, and digitalizing and decoding the amplified signal to be readable by a computer 3 through a serial port, and the computer 3 for reading the brain waves coded by the interface unit 2, and after verifying a psychological state of the subject by continuously verifying the brain waves through a Hidden Markov Model (HMM) which is used in a statistical signal process, displaying the results as a chromatic bar 4 designated by the subject on a monitor. The interfacing unit 2 may be separately provided as a peripheral of the computer or a built-in component of the computer.

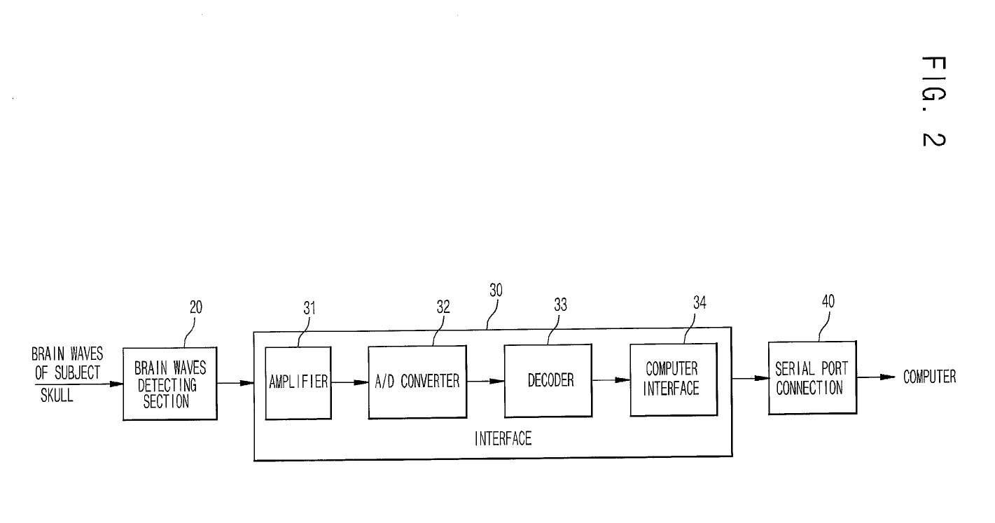

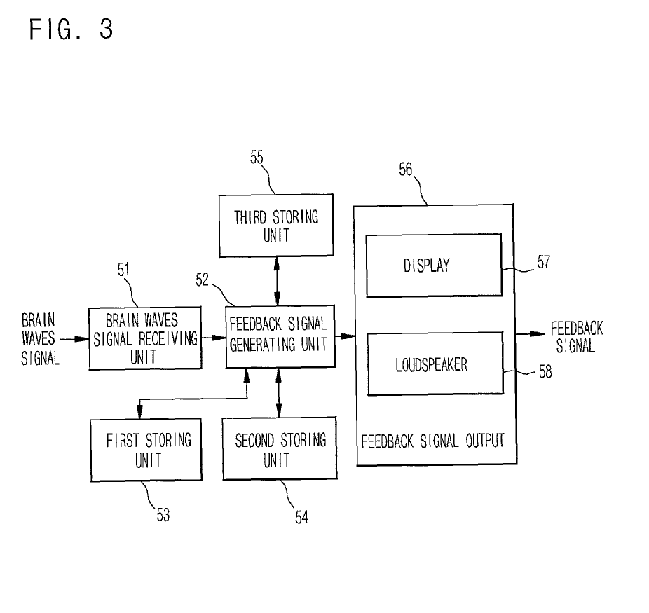

The construction as described above will now be explained in detail with reference to FIGS. 2 and 3.

FIG. 2 is a block diagram illustrating the construction of the brain waves measuring unit of FIG. 1, and FIG. 3 is a block diagram illustrating the construction of the biofeedback unit embedded in the computer system of FIG. 1.

As shown in FIG. 2, the brain waves measuring unit 1 comprises a brain waves detecting section 20, an interface 30, and a serial port connection 40.

The brain waves detecting section 20 detects at least one channel of a brain waves signal at desired points on a skull of the subject by use of a number of electrodes, and employs a headband to easily detect the brain waves on a frontal lobe.

The interface 30 is connected to the brain detecting section 20 via wire or wireless, and preferably, uses a shielded cable to prevent a noise from being happened. The interface 30 comprises an amplifier 31, an analog-to-digital (A/D) converter 32, a decoder 33, and a computer interface 34.

The amplifier 31 amplifies feeble signals of brain waves detected by the brain detecting section 20 to a certain level of signals, and includes a filter to control the noise contained in the amplified signals. The A/D converter 32 samples a number of channels of brain waves signal amplified by the amplifier at certain intervals to convert the brain waves to a digital value.

The coder 33 sequentially codes the digital signal of the brain waves sampled by the AD converter 32 as an identifier of each channel and a certain bite of digital value by each channel in real time.

The computer interface 34 transfers the coded digital signal from the coder 33 to a serial port of the computer 3.

The serial port connection 40 is connected to the interface 30, and transfers a digital decoding value for the brain waves signal outputted from the computer interface 34, which is connected to the serial port of the computer, of the interface to the computer. The serial port connection 40 includes an RS-232C.

The computer 3 comprises, as shown in FIG. 3, a brain waves signal receiving unit 51 for receiving the brain waves signal transferred from the interfacing unit 2 (FIG. 2) via the serial port, a first storing unit 53 for storing the detected brain waves signal of the subject, a second storing unit 54 for storing a chromatic data indicative of the psychological state, of which a color tone related with the psychological state is optionally designated by the subject, a third storing unit 55 for storing reference model values related with the psychological state of the subject, and a feedback signal generating unit 52 for storing the brain waves signal received from the brain waves signal receiving unit 51 in the first storing unit 53, analyzing the stored brain waves signal to extract parameters, comparing a model value of the extracted parameter with the reference model value stored in the third storing unit 55 to verify the present psychological state of the subject, and outputting a chromatic bar information corresponding to the psychological state stored in the second storing unit 54 on a display 57 of a feedback signal output 56 according to the present psychological state of the subject. As described above, although the present psychological state of the subject is displayed in the form of the chromatic bar information, it may be displayed in the type of sound values through a loudspeaker 58. Hereinafter, it will be explained with reference to the embodiment of displaying the chromatic bar.

The feedback signal generating unit 52 which is a major part of the present invention will now be explained in detail.

The values of the brain waves, which is reflective of the psychological state of the subject, detected by the brain waves measuring unit 1 is stored in the first storing unit 53 (FIG. 2). The psychological state comprises a stable and quiet state, a sad or anxious state, a happy state or the like, and is not limited thereto.

If the brain waves signal of the subject is detected, the subject maps the detected brain waves signal related with the psychological state of the subject as a desired color. After the mapping process, the mapped color information, that is, the color information related with the psychological state, is stored in the second storing unit 54. The subject selects one specific color, and designates each psychological state to another color having a depth different from that of the specific color. The designated colors are stored in the second storing unit 54. For example, if a green color is selected as the psychologically changed state, a stressful state is represented by a light green, while a relaxed state is represented by a sea green. A boundary color between them may be selected by the subject. The chromatic bar information indicative of a depth variation of the color obtained by the above method is stored in the second storing unit 54.

The feedback signal generating unit 52 stores a model which is an index to verify each psychological state, i.e., the Hidden Markov Model. Signal process of the recorded brain waves is executed by the model, and the signal process will now be explained.

A data of a certain length recorded in a specific psychological state is continuously cut several frames capable of easily processing the data. Feature information is extracted from each frame. At that time, the extracting process uses specific information so-called an autoregressive parameter.

In order to make several parameters obtained by the above process in a brief and simple form, parameters having analogous values are put together, thereby being represented as a single group. The representation of the single group is performed by a vector quantization method using a K-means algorism.

The data concerned with the brain waves is quantized to establish a code book through the vector quantization method using the K-means algorism, and then a code series obtained from the method computes Hidden Markov Model, which will be stored in the third storing unit 55.

The relevant model by each psychological state is created through the method, and the number of the models created thus is identical with that of the psychological states of the subject.

In case that the Hidden Markov Model by each psychological state is stored, the process in which the subject utilizes the system of the present invention to verify his or her psychological state and wish to stably convert the present psychological state will now be explained.

The brain waves are measured by the brain waves measuring unit 1 shown in FIG. 1. Specifically, after the brain waves of the subject is detected by the brain waves detecting section 20, the detected micro brain waves signal is amplified to a certain level by the amplifier 31. At that time, the noise signal contained in the brain waves signal is eliminated by the filter.

The brain waves signal with the noise signal eliminated is sampled by the A/D converter 32 at certain intervals to convert the brain waves signal to the digital value. The converted signal is coded by the coder 33 sequentially as an identifier of each channel and a certain bite of digital value by each channel in real time.

The digital value of the brain waves is transferred to the computer via the computer interface 34 and the serial port connection 40.

And then, the brain waves signal receiving unit 51 of the computer 3 receives the brain waves signal transferred from the interfacing unit 2 via the serial port, and supplies the received brain waves signal to the feedback signal generating unit 52.

The feedback signal generating unit 52 stores the measured brain waves signal of the subject received via the interfacing unit 2 in the first storing unit 53. After the subject maps the detected brain waves signal related with the psychological state of the subject as a desired color, the feedback signal generating unit 52 stores the mapped color information in the second storing unit 54. The mapping process is described hereinbefore, and the detailed description thereof will be omitted. The color information stored in the second storing unit 54 is a chromatic bar information indicative of the depth variations of color.

The feedback signal generating unit 52 stores the Hidden Markov Model which is an index to verify each psychological state in the third storing unit 55. If the measured brain waves signal of the subject is inputted, the feedback signal generating unit 52 compares the Hidden Markov Model with a new Hidden Markov Model related to the measured brain waves signal of the subject, and displays the chromatic information (bar information) related to the present psychological state of the subject on the display 57.

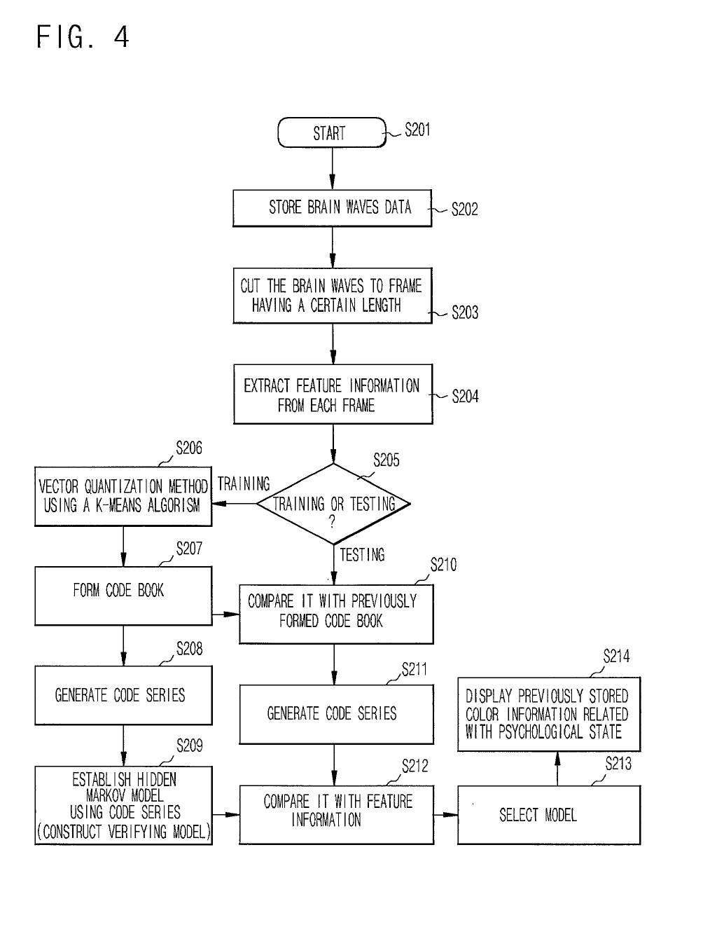

The chromatic display-type biofeedback method using the brain waves according to the present invention corresponding to the chromatic display-type biofeedback system using the brain waves according to the present invention will now be explained with reference to FIG. 4.

FIG. 4 is a flowchart of a chromatic display-type biofeedback method using the brain waves according to the present invention.

When the subject feels anxious or stress, the brain waves of the subject is measured by a headband-type brain measuring unit with a number of sensors (not shown) attached thereto, and the measured brain waves signal related to the present psychological state is stored (step S201). At that time, although it is determined that the subject is in a relaxed and quiet state, the brain waves of the subject can be measured and stored with a same method as that described above.

Of course, even though two cases are illustrated as described above, it is not limited to other psychological states that the subject want to remember: the subject is in a sad or anxious state; the subject is in a happy state or the like.

And then, the subject maps the detected brain waves signal related with the psychological state of the subject as a desired color. If the subject selects one specific color, the subject designates each psychological state to another color having a depth different from that of the specific color. For example, if a green color is selected as the psychologically changed state, the stressful state is represented by a light green, while the relaxed state is represented by a sea green. A boundary color between them may be selected by the subject.

The chromatic bar information indicative of a depth variation of the color obtained by the above method is stored. After all, the brain waves pattern related with the specific psychological state of the subject is designated by the subject as a desired color, thereby forming the chromatic bar.

A model which is an index to verify each psychological state of the subject has to be stored separately from the chromatic bar information.

The model is obtained by signal processing the stored brain waves, and the signal process will now be explained.

The system continuously cuts a brain waves data of a certain length measured at a specific psychological state by a unit of a very short length so-called a frame capable of easily processing the data (step S203).

And then, a feature information is extracted from each frame (step S204). At that time, the extracting process uses a feature information so-called an autoregressive parameter.

In order to make several parameters obtained by the above process in a brief and simple form, parameters having analogous values are put together, thereby being represented as a single group. The representation of the single group is performed by a vector quantization method using a K-means algorism (step S206).

The data concerned with the brain waves is quantized to establish a code book through the vector quantization method using the K-means algorism (step S207), and then a code series is generated according to the code book (step S208).

After generating the code series, a reference verifying mode so-called the Hidden Markov Model is constructed (step S209).

The reference modes according to several psychological states of the subject are constructed with the above method, and they are stored in the storing unit. The number of models is identical with that of the psychological states of the subject.

In case that the Hidden Markov Model by each psychological state is stored, the process in which the subject utilizes the system of the present invention to verify his or her psychological state and wish to stably convert the present psychological state will now be explained.

In case of establishing a reference model according to several psychological states of the subject, if the subject wishes to put his or her mind at rest by use of the system of the present invention, the brain waves of the subject is firstly measured and stored.

The feature information by each frame related with the brain waves corresponding to the present measured psychological state of the subject is obtained, and is compared with the previously extracted feature information. In other words, the feature information is extracted, and is compared with the code book established previously (step S210) to generate a code series (step S211).

The code series generated by the above process is compared with the feature information indicative of the models by each psychological state (step S212), and one model which is determined to be very similar to the feature information (step S213).

After selecting one model, it is considered that the present psychological state of the subject is identical to the psychological state represented by the model, and the color (chromatic bar) information designated to that psychological state is outputted through the monitor (step S214).

Therefore, by watching the color displayed as described above, the subject can monitor in real time the situation that the psychological state of the subject is changed. Upon repeatedly remind the subject of the color of high or low depth, the psychological state of the subject may be converted to the designated psychological state, i.e., a stable state.

With the system and method according to the present invention, after the brain waves generated from the subject, which are indicative of anxious or relaxed psychological state, are recoded by himself or herself, the subject transforms each psychological state into a chromatic bar represented by a desired color. After that, when the subject measures his or her brain waves, a specific color is displayed on a monitor, so that the subject may verify the present psychological state and repeatedly remind him or her of a color designated as a relaxed state to induce the reaching of the relaxed state. At that time, after a psychological state of the subject is verified by modeling the brain waves using a Hidden Markov Model (HMM) which is used in a statistical signal process, the results is displayed as a chromatic bar designated by the subject on a monitor, so that conversion of the relevant psychological state can be rapidly and easily achieved.

Therefore, the present invention can be employed for persons who are exposed to a stressful working environment during a long time, are in need of a rest, or try to relax the psychological state before starting the work demanding of attention.

The forgoing embodiment is merely exemplary and is not to be construed as limiting the present invention. The present teachings can be readily applied to other types of apparatuses. The description of the present invention is intended to be illustrative, and not to limit the scope of the claims. Many alternatives, modifications, and variations will be apparent to those skilled in the art.

What is claimed is:

1. A chromatic display-type biofeedback method using brain waves, the method comprising the steps of:
measuring and storing the brain waves of a subject to verify a psychological state of the subject;
forming a chromatic bar by designating each psychological state of a brain waves pattern related with a specific psychological state of the subject as a desired color;
establishing a reference model according to a plurality of psychological states of the subject;
dividing a brain waves data measured at a specific psychological state of the subject at a frame of a certain dimension, and extracting a feature information from each frame;
comparing the extracted feature information with the preset feature information of the brain waves related with a reference model according to the psychological states; and
selecting one model indicative of the psychological state of the subject for the feature information similar to each other, and displaying a previously stored chromatic bar information corresponding to the mode.

2. The method as claimed in claim 1, wherein the reference model establishing step comprises the steps of:
measuring the brain waves to verify the psychological state of the subject;
dividing the measured brain waves data at a unit of a frame of a desired dimension, and extracting a parameter value for a feature information from each frame; and
sorting each parameter having an analogous value among the parameters extracted from each frame to construct the brain wave pattern according to the psychological state, and establishing a reference model according to each brain waves pattern.

3. The method as claimed in claim 2, wherein the parameter is an autoregressive parameter.

4. The method as claimed in claim 2, wherein the reference model establishing step comprises the steps of:
sorting each parameter having an analogous value among the parameters extracted from each frame as a single group to form a code book; and
establishing a Hidden Markov Model by use of each code series obtained by applying the code book to a training data.

5. The method as claimed in claim 4, wherein the code book is established by a vector quantization method using a K-means algorism.

6. The method as claimed in claim 1, wherein in the chromatic bar information displaying step, a color of the chromatic bar is changed by variation of the brain waves of the subject.

7. A chromatic display-type biofeedback system using brain waves, the system comprising
a brain waves measuring unit for measuring the brain waves to verify a psychological state of a subject;
a first storing unit for storing a chromatic bar formed by designating each psychological state of a brain waves pattern related with a specific psychological state of the subject as a desired color;
a second storing unit for storing reference models related with a plurality of psychological states of the subject; and
a feedback signal generating unit for a) dividing a brain waves data measured at a specific psychological state of the subject at a frame of a certain dimension, extracting a feature information from each frame, and comparing the extracted feature information with the storing reference models related with a plurality of psychological states stored in the second storing unit, and b) selecting one model indicative of the psychological state of the subject for the feature information similar to each other, and displaying a previously stored chromatic bar information corresponding to the mode.

8. The system as claimed in claim 7, wherein the feedback signal generating unit includes:
a parameter extracting section for dividing the measured brain waves data at a unit of a frame of a desired dimension, and extracting a parameter value for a feature information from each frame; and
a model establishing section for sorting each parameter having an analogous value among the parameters extracted from each frame as a single group through a vector quantization method using a K-means algorism to form a code book, and establishing a Hidden Markov Model by use of each code series obtained by applying the code book to a training data.

9. A storage medium for executing a chromatic display-type biofeedback method using brain waves, the storage medium capable of being read by a digital processor, and storing a program of commands executed by the digital processor, the program being implemented by types, with the program comprising the steps of:
measuring and storing the brain waves of a subject to verify a psychological state of the subject;
forming a chromatic bar by designating each psychological state of a brain waves pattern related with a specific psychological state of the subject as a desired color;
establishing a reference model according to a plurality of psychological states of the subject;
dividing a brain waves data measured at a specific psychological state of the subject at a frame of a certain dimension, and extracting a feature information from each frame;
comparing the extracted feature information with the preset feature information of the brain waves related with a reference model according to the psychological states; and
selecting one model indicative of the psychological state of the subject for the feature information similar to each other, and displaying a previously stored chromatic bar information corresponding to the mode.
